(19) United States
(12) Patent Application Publication
(10) Pub. No.: US 2019/0192290 A1
(43) Pub. Date: Jun. 27, 2019

(54) CATHETER WITH RING STRUCTURE FOR RE-SHEATHING OF SELF-EXPANDING IMPLANTS

(71) Applicant: BIOTRONIK AG, Buelach (CH)

(72) Inventors: Alois Pfenniger, Biel (CH); Andreas Bitzer, Zuerich (CH)

(21) Appl. No.: 16/225,664

(22) Filed: Dec. 19, 2018

(30) Foreign Application Priority Data
Dec. 21, 2017 (EP) 17209494.8

(51) Int. Cl. A61F 2/24

(52) U.S. Cl. CPC A61F 2/2418; A61F 2/2409; A61F 2/954; A61F 2/2436; A61F 2/243

(57) ABSTRACT

A catheter device for implanting an implant includes a self-expandable framework and an outer shaft having a capsule at a distal end. The capsule surrounds the framework such that they are displaceable relative to one another, which permits the framework to be released in sections. A released portion of the framework expands automatically and can be re-introduced into the capsule. A ring structure is displaceable in the distal direction (R′) beyond the capsule, such that an already released, expanded portion of the framework is contactable by the ring structure to limit axial forces exerted on the capsule and facilitate re-sheathing. The framework can be a self-expanding stent that supports a heart valve prosthesis.

### PRIORITY CLAIM

This application claims priority under 35 U.S.C. § 119 and all applicable statutes and treaties from prior European Application EP 17209494.8, filed Dec. 21, 2017.

### FIELD OF THE INVENTION

A field of the invention is catheters for implants, and particularly heart valve prostheses. A particular application is a catheter device for implanting a self-expanding heart valve prosthesis, but the invention provides catheters suitable for any self-expanding implant which, after partial release, is to be compressed again to its original diameter.

### BACKGROUND

Catheter devices of this kind are used for example for implanting heart valve prostheses, for example in the case of transcatheter aortic valve implantation.

Heart valve prostheses generally include a heart valve which is made for example from a biological material and is fixed to a stent framework, which is used as a carrier of the heart valve and in particular for the later anchoring of the heart valve in the heart. The stent frameworks can be self-expanding.

With self-expansion of the stent framework, the stent framework expands to the diameter of the diameter of the heart valve prosthesis in the radial direction, i.e. in a plane oriented perpendicularly to the longitudinal axis or axial direction of the catheter device.

As shown in FIGS. 1A-1C, this can be problematic insofar as the diameter D of the portion of the heart valve prosthesis or of the stent framework already released can significantly exceed the diameter D′ of the capsule 4 in the radial direction. When re-inserting (resheathing) the heart valve prosthesis 2 into the capsule 4, the diameter of the part T which has already been unfolded must be reduced again substantially to the original diameter. Here, during the re-insertion, the heart valve prosthesis 2 or the associated stent framework 3 is deformed in an umbrella-like manner, in which the diameter D of the stent framework or of the heart valve prosthesis decreases in the radial direction over a short axial distance A to the capsule diameter at the distal end of the capsule 4. A re-insertion of the heart valve prosthesis into the capsule is necessary, for example, when the positioning of the partially released implant has to be corrected or the implantation has to be terminated for other reasons and the heart valve prosthesis has to be removed again from the body.

Here, via its distal edge, which surrounds an opening of the capsule via which the heart valve prosthesis can be retracted back into the capsule, the capsule exerts axial forces (i.e. forces acting in the direction of the longitudinal axis of the catheter device) of corresponding magnitude onto the stent framework or the heart valve prosthesis, which forces load the stent framework/heart valve prosthesis and also the capsule and hinder the re-insertion (what is known as resheathing). Here, it can also be that the heart valve prosthesis can no longer be retracted fully into the capsule, whereby the part of the heart valve prosthesis not covered by the capsule protrudes radially outwardly. In a state of this kind the heart valve prosthesis can be neither implanted nor removed safely from the body.

### SUMMARY OF THE INVENTION

A preferred catheter device includes a self-expandable framework, which in particular preferred embodiments is a self-expanding stent that supports a heart valve prosthesis. An outer shaft has a capsule at a distal end, which capsule surrounds the framework. The capsule and the framework are displaceable relative to one another, such that the framework is releasable in part, wherein a released portion of the framework expands automatically and can be completely re-introduced into the capsule. A ring structure is positionable over an already released, expanded portion of the framework (such that the ring structure surrounds this portion), wherein the ring structure is displaceable in the distal direction, such that the already released, expanded portion of the framework is contactable and/or compressible by the ring structure and is guidable by the ring structure as the framework is re-inserted into the capsule. The ring structure can be arranged radially over the capsule (such that the ring structure surrounds the capsule).

### BRIEF DESCRIPTION OF THE DRAWINGS

Further features, advantages and embodiments of the invention will be explained in detail hereinafter with reference to the drawings. In the drawings:

FIGS. 1A-1C show the disadvantageous umbrella form of the stent framework or of the heart valve prosthesis occurring with resheathing in the prior art;

FIGS. 2A-2D show schematic views of a catheter device according to the invention;

FIGS. 3A-3E show views of a ring structure according to the invention in the form of a one-piece self-expandable ring;

FIGS. 4A-4E show views of a ring structure according to the invention which includes a plurality of ring elements;

FIG. 5 shows a perspective view of a ring structure according to the invention formed from a plurality of ring elements which are connected to one another;

FIGS. 6A-6B shows views of a ring structure according to the invention formed from a plurality of ring elements which engage with one another;

FIGS. 7A-7B shows perspective views of a joint of a catheter device according to the invention;

FIG. 8 shows a perspective view of a detail of a catheter device according to the invention with separate ring elements of a ring structure;

FIG. 9 shows a perspective view of an outer jacket for holding a ring element or the bars of a ring element of a ring structure according to the invention;

FIG. 10 shows a perspective view of a device for moving ring elements of a ring structure according to the invention that are arranged in an axially offset manner;

FIGS. 11A-11C show various views of an embodiment of the invention, in which the ring structure allows a compression of the partially expanded heart valve prosthesis/stent framework;

FIGS. 12A-12C show various views of a modification of the embodiment shown in FIGS. 11A-11D;

FIGS. 13A-13D show, in steps a to d, a re-insertion (resheathing) of a partially expanded or released heart valve prosthesis, wherein in particular the already expanded portions of the heart valve prosthesis or of the stent framework are actively compressed by means of the ring structure (for example in the manner of FIGS. 11, 12, 15 and 16) so as to facilitate the re-insertion into the capsule 4;

FIGS. 14A-14B show various views of an embodiment of the invention, in which the capsule itself forms a ring structure reducible in diameter;

FIGS. 15A-15D show a further embodiment of the invention in which the ring structure includes bars and in particular helically curved and forcibly guided connection bars; and

FIGS. 16A-16D show a schematic depiction of the operating principle of the embodiment according to FIGS. 15A-15D.

### DETAILED DESCRIPTION OF THE PREFERRED EMBODIMENTS

Preferred embodiments are illustrated including a self-expanding stent framework that supports a heart valve prosthesis. In accordance with one embodiment of the invention, the ring structure is displaceable beyond the capsule in the distal direction, such that an expanded portion of the heart valve prosthesis (in particular of the stent framework) that has already been released is contactable by means of the ring structure.

In the present description, “distal” means that a corresponding distal component, a distal portion or a distal end, in the axial direction of the outer shaft along which the outer shaft or the longitudinal axis of the outer shaft extends, is distanced further from a handgrip or an operator (physician) of the catheter device than a proximal component, a proximal portion or a proximal end. Accordingly, the distal direction along the catheter device points from proximally to distally, and the proximal direction accordingly points along the catheter device from distally to proximally. The radial direction in each case is perpendicular to the axial direction.

The stent framework is preferably self-expanding. In other words, it includes a material or consists of a material that permits a compression of the stent framework in the radial direction to a reduced diameter, wherein the stent framework experiences an automatic unfolding or expansion in the radial direction to the original diameter as soon as the stent framework is no longer subjected to any external restriction of its diameter (for example when the stent framework is released from its capsule surrounding the stent framework). Here, the heart valve, which is fixed to the stent framework and which in particular may be a biological heart valve, is also unfolded or relaxed. Suitable materials that have these properties are shape-memory materials (for example nickel-titanium alloys, such as nitinol).

In a preferred embodiment of the invention the heart valve prosthesis is arranged in the distal portion of a catheter shaft which is displaceable relative to the outer shaft. The heart valve prosthesis is connected here releasably to this catheter shaft (also referred to hereinafter as the inner shaft) up to the complete release of the prosthesis.

In accordance with a preferred embodiment of the invention the ring structure is designed to limit a diameter of the stent framework at the location of the ring structure such that an axial force which is exerted onto the capsule as the heart valve prosthesis is resheathed into the capsule is limited accordingly. The ring structure, during the resheathing, reduces the diameter of the stent and thus facilitates the resheathing of the heart valve prosthesis into the capsule.

In accordance with one embodiment of the invention the ring structure is designed to be lockable in position in such a way that it retains its physical position in relation to the capsule as the heart valve prosthesis is resheathed into the capsule. The ring structure can thus take up axial forces as the heart valve prosthesis is resheathed into the capsule and in particular is designed to already limit the diameter of the stent framework outside the capsule as the heart valve prosthesis is resheathed into the capsule, such that excessive (umbrella-like) deformation of the stent framework or of the heart valve prosthesis during the resheathing is prevented.

In accordance with one embodiment of the invention the catheter device also has a plurality of bars for supporting the ring structure, which bars are each connected to the ring structure via a first end portion. The bars do not necessarily have to be formed in one piece, and instead can be composed of individual portions. Bars can thus include portions that for example are compressible in the axial direction, for example in the form of a spring (see also below).

In accordance with one embodiment of the invention the bars are configured to displace the ring structure in the axial or distal direction beyond the capsule, such that the ring structure can contact the partially unfolded heart valve prosthesis (in particular the stent framework) in the above-described way.

In accordance with one embodiment of the invention the catheter device has an outer stabilisation shaft for stabilising the outer shaft, wherein the outer shaft is arranged in a lumen of the stabilisation shaft surrounded by the stabilisation shaft, and wherein the outer shaft and the stabilisation shaft are displaceable relative to one another.

With regard to the bars, it is also provided in one embodiment of the invention that these are each fixed to a distal end of the stabilisation shaft via a second end portion, which is opposite the corresponding first end portion in the axial direction. The ring structure can therefore move via the bars and the stabilisation shaft in the direction of the capsule fixed to the outer shaft, or beyond the capsule.

In one embodiment of the invention, where the bars are fixed to the distal end of the stabiliser shaft, the stabiliser shaft is axially displaceable relative to the outer shaft and relative to the heart valve prosthesis (and thus the inner shaft). Here, it is irrelevant whether one or more further catheter shafts is/are also arranged between the outer shaft, which holds the heart valve prosthesis in the radially compressed state, and the stabiliser shaft. One or more further catheter shafts can likewise also be arranged radially outside the stabiliser shaft. In this embodiment, there is axial displaceability between stabiliser shaft on the one hand and outer and inner shaft on the other hand. It is also irrelevant which catheter shaft is movable in which direction; what is key is merely the movability thereof relative to one another. The outer shaft can preferably be retracted proximally, and the stabiliser shaft advanced distally, whilst the inner shaft is not moved.

The catheter device, in accordance with one embodiment of the invention, also includes an inner shaft, which is arranged in a lumen of the outer shaft, wherein a carrier is provided at a distal end of the inner shaft and supports the heart valve prosthesis. The outer shaft and inner shaft in accordance with one embodiment are preferably displaceable relative to one another, such that the carrier can be removed from the capsule in order to release the heart valve prosthesis or in order to release the heart valve prosthesis in sections. A fastening structure can be provided on the carrier, which fastening structure is engaged with a part of the stent framework that for example is provided on a portion of the stent framework that is released last, such that a heart valve prosthesis released merely in part or in sections can be withdrawn back into the capsule (by a corresponding relative movement of the carrier and the capsule).

Furthermore, in one embodiment the inner shaft can include a lumen in which a guide wire runs, such that the inner shaft (with carrier), the outer shaft (with capsule), and the stabilisation shaft are guidable by the guide wire.

In accordance with one embodiment of the invention, the second end portions of the bars are connected via a joint to the distal end of the stabilisation shaft. In particular in the case of transcatheter aortic valve implantation (TAVI for short), the catheter device is exposed to significant bending deformations (in particular in the aortic arch). By means of the joint, the different lengths of the curved bars relative to the middle line of the catheter device can be compensated advantageously.

With regard to the joint, it is provided in accordance with one embodiment of the invention that the joint includes a first joint ring, a second joint ring and a third joint ring, wherein the second joint ring is arranged between the first and the third joint ring, and wherein the three joint rings surround the outer shaft in the circumferential direction of the outer shaft, wherein the first joint ring is connected to the second joint ring so as to be tiltable about a first axis, and wherein the second joint ring is connected to the third joint ring so as to be tiltable about a second axis, wherein the two axes run orthogonally to one another. Furthermore, in order to connect this joint structure to the stabilisation shaft or to the bars, it is provided that the first joint ring is rigidly connected to the second end portions of the bars, and that the third joint ring is rigidly connected to the distal end of the stabilisation shaft.

The above-described embodiment with a joint consisting of 3 joint rings ensures a tiltability in all spatial directions as a result of the orthogonal arrangement of the two axes. In one embodiment, where from the outset only the tiltability about one predefined axis is necessary, 2 joint rings are sufficient. Similarly, it can be advantageous for a finer adjustment of the tiltabilities of the axis to use more than 3 joint rings. The embodiment formed of 3 joint rings, however, is the optimum in respect of outlay alongside free movability of the arrangement.

In principle, various forms are conceivable for the ring structure. The ring structure for example can thus be formed in one piece or it can be composed of a number of elements.

In this regard it is provided in accordance with one embodiment of the invention that the ring structure is a continuous, closed, self-expanding ring.

In accordance with an alternative embodiment of the invention, the ring structure is formed by a plurality of ring elements, wherein the ring elements are movable relative to one another in such a way that the ring structure is movable from a first configuration into a second configuration, wherein the ring structure in the second configuration has a larger diameter than in the first configuration.

The ring structure can thus be transported advantageously in the first configuration by means of the catheter device to the implantation site, wherein an unfolding/expansion of the ring structure can then optionally follow the second configuration, in which the ring structure has a larger diameter and the partially released heart valve prosthesis can be suitably supported during the resheathing into the capsule (see above).

In the case of a ring structure in the form of a closed, one-piece ring, the bars can be suitably fixed to the ring via their first end portions (for example via a solder connection in each case).

For the case in which the ring structure is composed of separate ring elements, it is preferably provided in accordance with one embodiment that each bar is connected via its first end portion to an associated ring element. In particular, in each case two parallel bars can be connected to the same ring element, for example when the ring element is formed in a bight-like manner and has two ends accordingly (see below).

In accordance with one embodiment of the invention, it is also provided that every two adjacent ring elements engage in one another, such that the ring structure is formed by a chain of the ring elements, wherein in particular the ring elements or chain links are arranged such that they can be telescoped in the circumferential direction of the ring structure, such that the diameter of the ring structure is adjustable accordingly. In the first configuration the ring elements lie more closely together in the circumferential direction of the outer shaft than in the second configuration.

The chained ring elements can each be formed in particular in a bight-like shape, i.e. they each have a curved portion and two mutually opposed ends which are distanced from one another in the circumferential direction of the outer shaft. The bars can adjoin these ends, wherein the bars in each case can then be connected integrally to the ring elements or ends thereof.

In accordance with one embodiment of the invention, the individual ring elements are not engaged with one another, and instead are arranged adjacently, thus forming the ring structure. In this regard it is provided in accordance with one embodiment that each two adjacent ring elements in the first configuration are arranged axially offset in relation to one another, such that the ring structure includes first ring elements which in the first configuration of the ring structure are arranged in front of second ring elements of the ring structure in the distal direction. In accordance with one embodiment, it is also provided that in the second configuration all ring elements are arranged adjacently in the circumferential direction and in particular are disposed in the same position and at the same level in the axial direction and distal direction respectively.

Here, the corresponding ring element can again be bight-shaped, in particular shaped in the form of a closed bight. In other words, the corresponding ring element again has a curved portion and two mutually opposed ends, which in particular bear against one another.

In the case of these ring elements it is thus preferably provided that a width of the corresponding ring element or of the corresponding bight in the circumferential direction of the ring structure away from the ends of the ring element is greater than the width of the ring element or the corresponding bight in the circumferential direction at the ends of the ring element. With a corresponding axially offset arrangement of adjacent ring elements, the diameter of the ring structure as a whole can hereby be reduced, since a ring element in the region of the ends of the adjacent (first) ring element can nestle against the adjacent ring element (or against the bars thereof).

It is furthermore provided in accordance with one embodiment of the invention that the bars connected to the first ring elements include a portion that is resiliently compressible in the axial direction and that can be formed for example by a spring, such that, when the ring structure is displaced beyond the capsule in the distal direction, the first ring elements, upon contact with the partially released heart valve prosthesis, are pushed back against a restoring force of the corresponding resiliently compressible portion until all ring elements have the same position in the axial direction. In this embodiment of the invention there is thus an automatic positioning of the ring elements in order to produce the second configuration of the ring structure by contact with the supported heart valve prosthesis.

Furthermore, in accordance with one embodiment of the invention, it can be provided that the bars are surrounded by an outer jacket surrounding the outer shaft or are fixed to an outer jacket surrounding the outer shaft. In particular, the outer jacket can be resilient and in particular can be radially extended up to a predefined diameter. In this way, the jacket can delimit a maximum diameter of the ring structure. Alternatively, the delimitation of the maximum diameter of the ring structure can also be implemented via wires, tapes or the like.

The bars can furthermore be fixed in particular to the jacket in that they penetrate the jacket at a number of points, such that they run alternately beneath and above the jacket.

The jacket can be made of a plastics fibre fabric, for example Dacron® fabric or another suitable material, in particular woven fabric. In principle, any fabric which has a limited extendibility in the circumferential direction is suitable here, wherein the term “fabric” is understood within the scope of the application to mean a composite of individual fibres.

In accordance with a further embodiment of the invention, it is provided that the heart valve prosthesis, in particular the stent framework, is actively compressible in diameter, i.e. in the radial direction, by means of the ring structure (alternatively or additionally).

It is thus provided in this embodiment that the catheter device has a ring structure which is displaceable in the distal direction, more specifically in particular over an already released, expanded portion of the heart valve prosthesis, such that this is compressible by means of the ring structure.

The ring structure to this end, in accordance with one embodiment of the invention, has a reducible diameter, such that the ring structure is contractible about the heart valve prosthesis, and in so doing can exert a force in the radial direction onto the heart valve prosthesis, which compresses the heart valve prosthesis, in particular the stent framework, in the radial direction, such that the heart valve prosthesis is retractable again into the capsule (resheathing).

In accordance with one embodiment, the ring structure includes a plurality of bars and an elongate flexible element. The bars each have a first end and a through-opening provided at the corresponding first end, wherein the elongate flexible element is guided through the through-opening.

The element can be a limp element. The elongate flexible element, which in particular is limp, is understood within the scope of this application to mean an element which is very easily bendable or movable in each spatial direction, but cannot be extended or stretched along its longitudinal axis. A limp flexible element of this kind can be, for example, a wire, a thread, or a cord. The element in particular forms a closed ring, such that a rotation of the bars about the longitudinal axis of the bars causes the flexible element to be wound around the respective bar, such that the ring structure is reduced in diameter, contracts around the partially expanded heart valve prosthesis, and compresses this in the radial direction for reinsertion into the capsule. The bars can be displaceable in the distal direction in particular by means of the stabilisation shaft, such that the ring structure is slidable by means of the stabilisation shaft over the partially expanded heart valve prosthesis and is reducible in diameter there. The bars can also be coupled via their second ends, which are opposite the first ends, to a mechanism by means of which the bars are rotatable about the respective longitudinal axis.

In accordance with one embodiment the first ends are spherical.

Furthermore, the ring structure can include an annular element, wherein the first ends of the bars each engage in an associated recess of the annular element and are mounted there rotatably about the longitudinal axis of the respective bar. The respective recess here forms a rotary bearing for the first end of the respective bar.

The recesses or plain bearings can in each case be arranged in a rigid portion of the annular element, wherein each two rigid portions adjacent in the circumferential direction are connected to one another by an intermediate portion, in particular a resiliently deformable intermediate portion. By rotating the bars, the resiliently deformable portions can be pressed together in the circumferential direction of the annular element, wherein the diameter of the annular element is reduced.

In accordance with a further embodiment it is provided that the ring structure is formed by the capsule itself.

Here, the capsule in accordance with one embodiment includes at least one, preferably at least three wall elements, wherein wall elements that are adjacent in the circumferential direction of the capsule overlap one another, and wherein each wall element includes a bearing for a first end portion of a bar, wherein each bar is rotatable in its bearing about its longitudinal axis, and wherein each bar has a through-opening, through which an elongate flexible element is guided.

The elongate, flexible element can again be a limp element (similarly to above). The element can also be a wire, a thread, or a cord. The element in particular forms a closed ring, such that a rotation of the bars about the longitudinal axis of the bars causes the flexible element to be wound around the respective bar, such that each two wall elements overlapping one another are slid one over the other in the circumferential direction, wherein the ring structure comprising the wall elements is reduced in diameter and in so doing contracts around the partially expanded heart valve prosthesis and compresses this in the radial direction for re-insertion into the capsule. The bars can also be coupled via their second end portions, which are opposite the first end portions, to a mechanism by means of which the bars are rotatable about the respective longitudinal axis. Embodiments having more than 3 wall elements function analogously to the previous description, similarly to embodiments having one or two wall elements. If, for example, just one wall element is used, this overlaps with itself. In other words, the circumference of this wall element is greater than that necessary for the diameter.

For wall elements, materials as are also used for catheter shafts (for example the outer shaft) are advantageous. These are plastics, such as polymers, in particular polyethylenes, such as HDPE.

In accordance with one embodiment, the ring structure includes a plurality of bars and an elongate flexible element. The bars each have a first end, at which the respective bar has a through-opening, wherein the elongate flexible element is guided through the through-openings. The first ends can each be spherical.

Furthermore, each first end is connected to a helically curved connection bar (in particular in one piece), wherein the respective connection bar has an angled end portion, which has a spherical end which has a through-opening, wherein an adjacent connection bar is mounted slidingly in the respective through-opening.

The elongate, flexible element can again be a limp element. The element can also be a wire, a thread, or a cord. The element forms a closed ring, such that a rotation of the bars about the longitudinal axis of the bars causes the flexible element to be wound around the respective bars, such that the ring structure is reduced in diameter, wherein the bars and the connection bars contract about the partially expanded heart valve prosthesis and compress this in the radial direction for re-insertion into the capsule. The bars can also be coupled via their second ends, which are opposite the first ends, to a mechanism by means of which the bars are rotatable about the respective longitudinal axis.

The ring structure and the associated optional bars, joint structures or elements can advantageously be surrounded by an additional thin jacket. This additional optional thin jacket or film protects the fabric at the implantation site against direct contact with the ring structure and accessories thereof.

Preferred embodiments advantageously allow a resheathing of the heart valve prosthesis, wherein the usual, umbrella-like deformation of the stent framework or of the heart valve prosthesis can be prevented. The forces acting axially on the stent framework in the event of re-insertion into the capsule are such that the heart valve prosthesis and the capsule are preserved accordingly, which has a positive effect on the long-term performance of the heart valve prosthesis.

Furthermore, the solution according to the invention is applicable to all conceivable catheter forms. The device according to the invention can thus also be provided as an auxiliary tool for an existing catheter system.

The above-described embodiments which allow a compression of the partially expanded heart valve prosthesis by means of the ring structure have a number of advantages. The space available in the rising aorta is usable by the expandable geometry. The limitations affecting the catheter system during the resheathing can hereby be reduced. The resheathing mechanism can be embodied as an auxiliary tool, such that it can be used on multiple (existing) catheter systems. Furthermore, in some embodiments axial forces are reduced, since the actuation of the mechanism is based on torsion. The risk of bulging of an elongate structure that is loaded in the axial direction is hereby avoided. The heart valve prosthesis, in particular the stent framework, can also be compressed by radial forces before it is retracted into the capsule. The loading of the heart valve prosthesis during the resheathing is hereby lower. All embodiments of the present invention however reduce the force that acts radially on the capsule during the re-insertion (resheathing) of the heart valve prosthesis, whereby the re-insertion of the heart valve prosthesis is simplified and made safer.

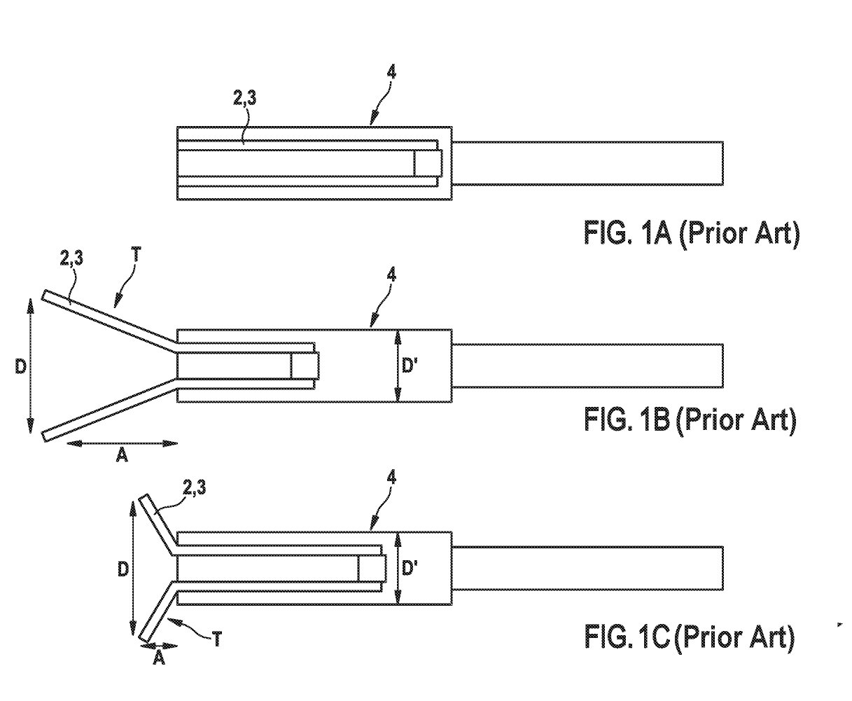

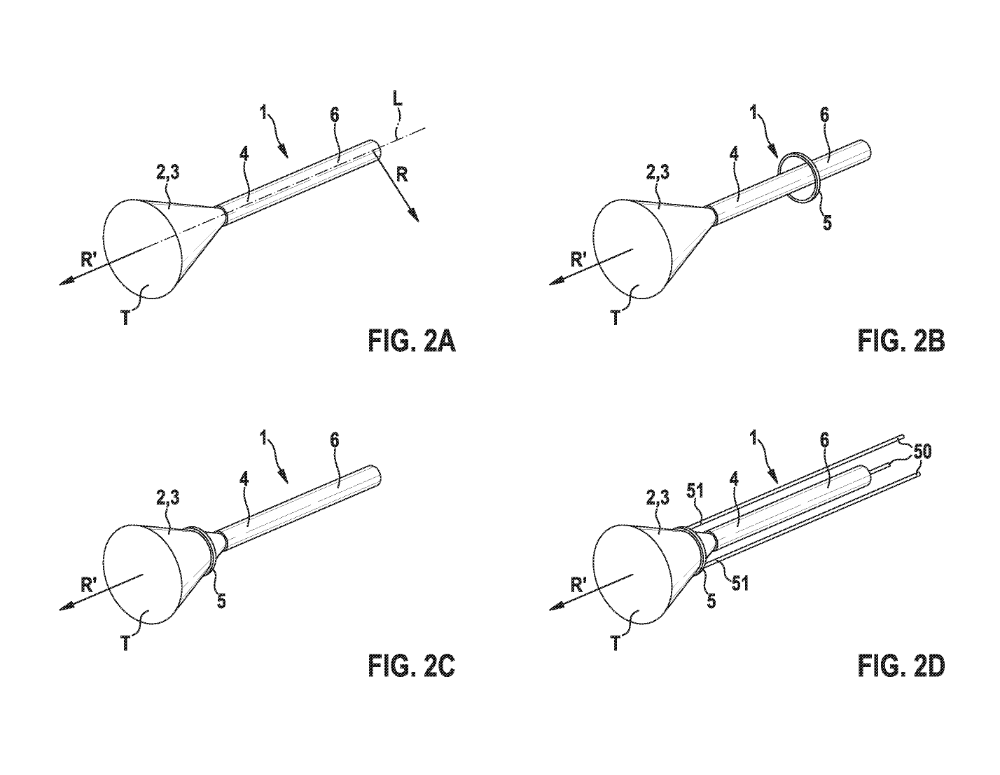

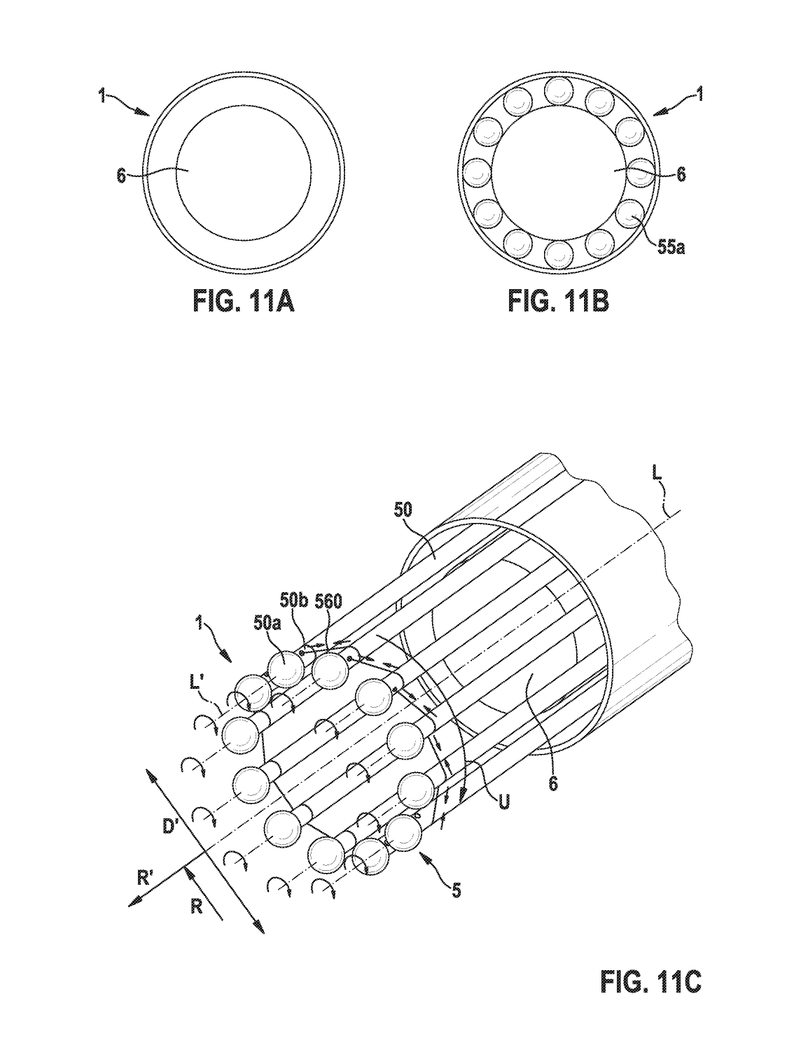

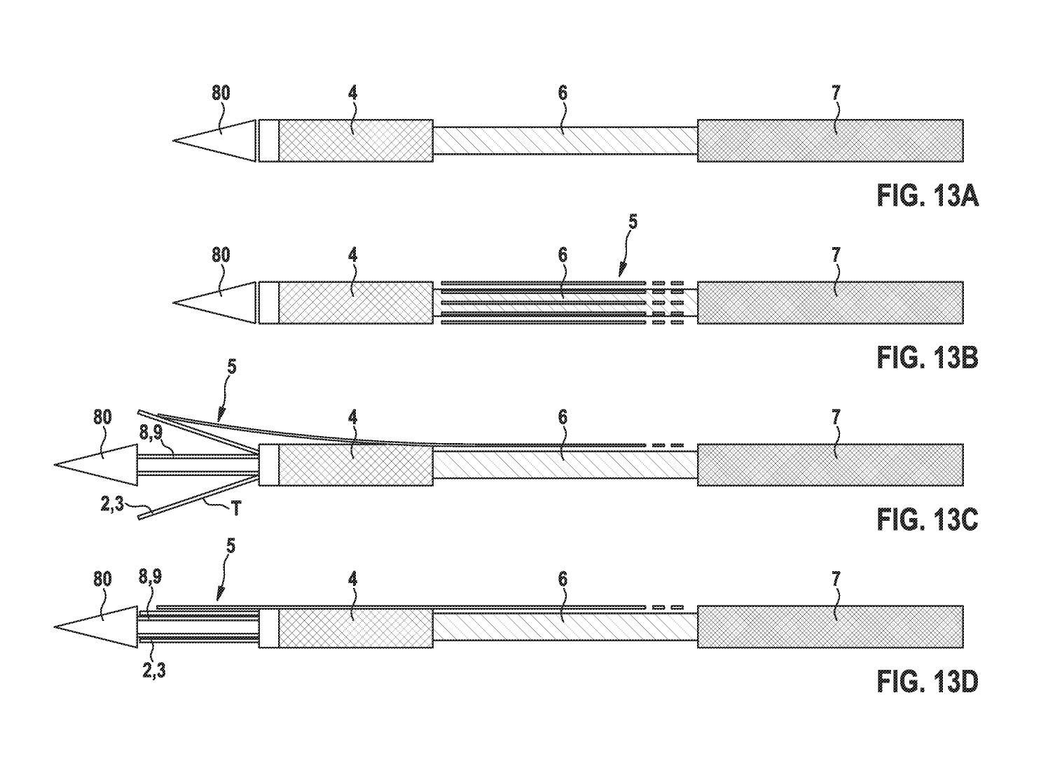

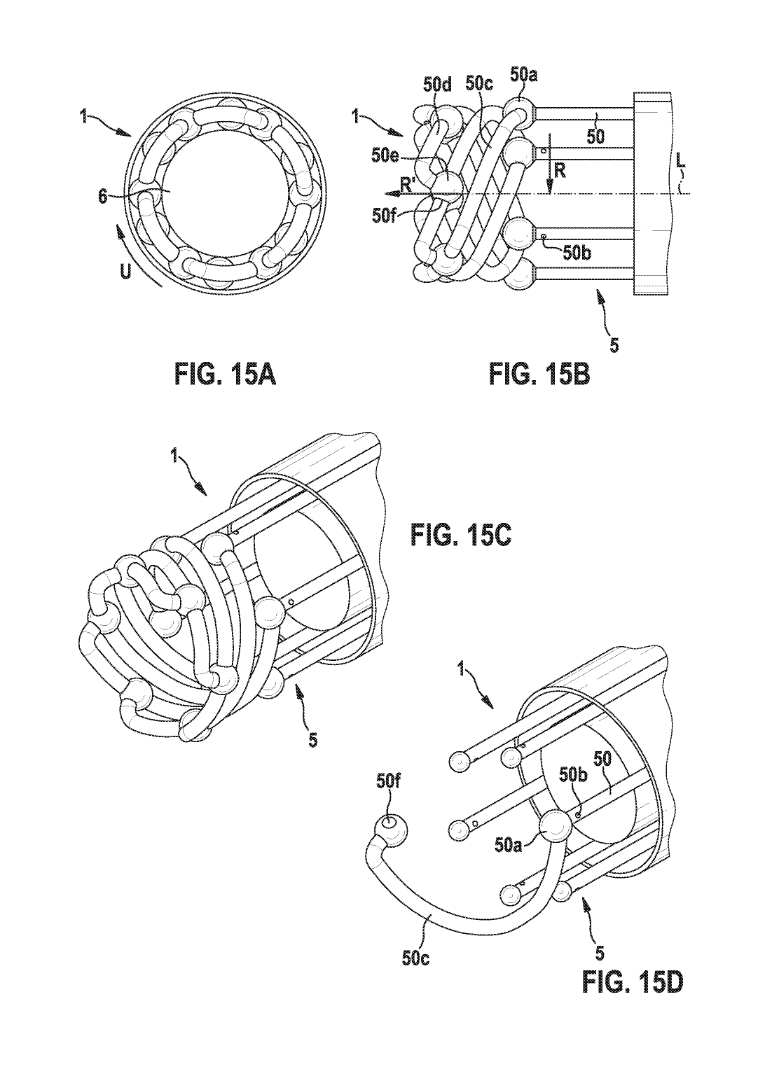

FIGS. 2A-2D show schematic views of a catheter device 1 according to the invention for implanting a heart valve prosthesis 2. The catheter device to this end includes a heart valve prosthesis 2, which has a heart valve, and a self-expandable stent framework 3, which supports the heart valve. To transport the heart valve prosthesis, the catheter 1 also includes an outer shaft 6 (see for example also FIGS. 13A-13D), which is connected at the distal end to a capsule 4, which surrounds the heart valve prosthesis 2. The distal end of the catheter device 1 (see also FIG. 13A-13D) forms the catheter tip 80 as distal end of the inner shaft 8, wherein the capsule 4 and the heart valve prosthesis 2/inner shaft 8 are displaceable relative to one another, such that the heart valve prosthesis is releasable in sections and is retractable into the capsule 4 (in particular completely). As soon as a portion T of the heart valve prosthesis 2 or the stent framework 2 is released, this unfolds, as shown FIGS. 1A-2D and FIGS. 13A-13D. In order to be able to retract the already unfolded portion T of the heart valve prosthesis 2 optionally completely into the capsule 4 (what is known as resheathing) without excessive umbrella-like deformation (see FIGS. 1A-1C) of the heart valve prosthesis 2 or of the stent framework 3, it is provided in accordance with the invention that the catheter device 1 includes an additional ring structure 5, which is displaceable in the distal direction R′ beyond the capsule 4, such that the already released, expanded portion T of the heart valve prosthesis (in particular of the stent framework) is contactable by means of the ring structure 5 and in particular is guidable in the event of re-insertion into the capsule 4 (in accordance with the embodiments described further below (FIGS. 11 to 16), the ring structure 5 is also designed for radial compression of the heart valve prosthesis 2 or of the stent framework 3).

The invention thus allows a re-insertion of the heart valve prosthesis 2 into the capsule 4 and a renewed release of the heart valve prosthesis 2 (for example if the heart valve prosthesis 2 was incorrectly positioned), without this leading to excessive loading of the heart valve prosthesis 2 or of the capsule 4 during the re-insertion into the capsule 4.

The catheter device 1 may also include an inner shaft 8, which is arranged in a lumen of the outer shaft 6, wherein a carrier 9 is provided by a distal end or the tip 80 of the inner shaft 8 (see also FIGS. 13A-13D) and supports the heart valve prosthesis 2. The outer shaft and inner shaft 6, 8 are in particular displaceable relative to one another, such that the carrier 9 can be removed from the capsule 4 in order to release or partially release the heart valve prosthesis 2. The outer shaft 6 can furthermore be guided in a lumen of a stabilisation shaft 7, which is used to stabilise the inner and outer shaft 8, 6. A fastening structure can also be provided on the carrier 9, which fastening structure is engaged with a part of the stent framework 3 that for example is provided on a portion of the stent framework 3 that is released last, such that a heart valve prosthesis 2 released merely in part or in sections can be withdrawn back into the capsule 4 (by a corresponding relative movement of the inner shaft 8/carrier 9 and the capsule 4). Furthermore, the inner shaft 8 can include a lumen in which a guide wire runs, such that the inner shaft 8 (with carrier 9), the outer shaft 6 (with capsule 4), and the stabilisation shaft 7 are guidable by the guide wire.

As can be seen in FIG. 2D, the catheter device 1, in order to hold or move the ring structure 5, includes a plurality of bars or longitudinally extended structures 50, which extend along the outer shaft 6 and in each case are connected to the ring structure 5 via a first end portion 51.

The bars are configured here to displace the ring structure 5 beyond the capsule 4 in the distal or axial direction L, such that the ring structure 5 can contact the partially unfolded heart valve prosthesis 2 (in particular the stent framework 3) in the above-described way and can guide it during the re-insertion into the capsule 4, wherein an excessive umbrella shape of the stent framework 3 at the capsule opening is prevented on account of the ring structure.

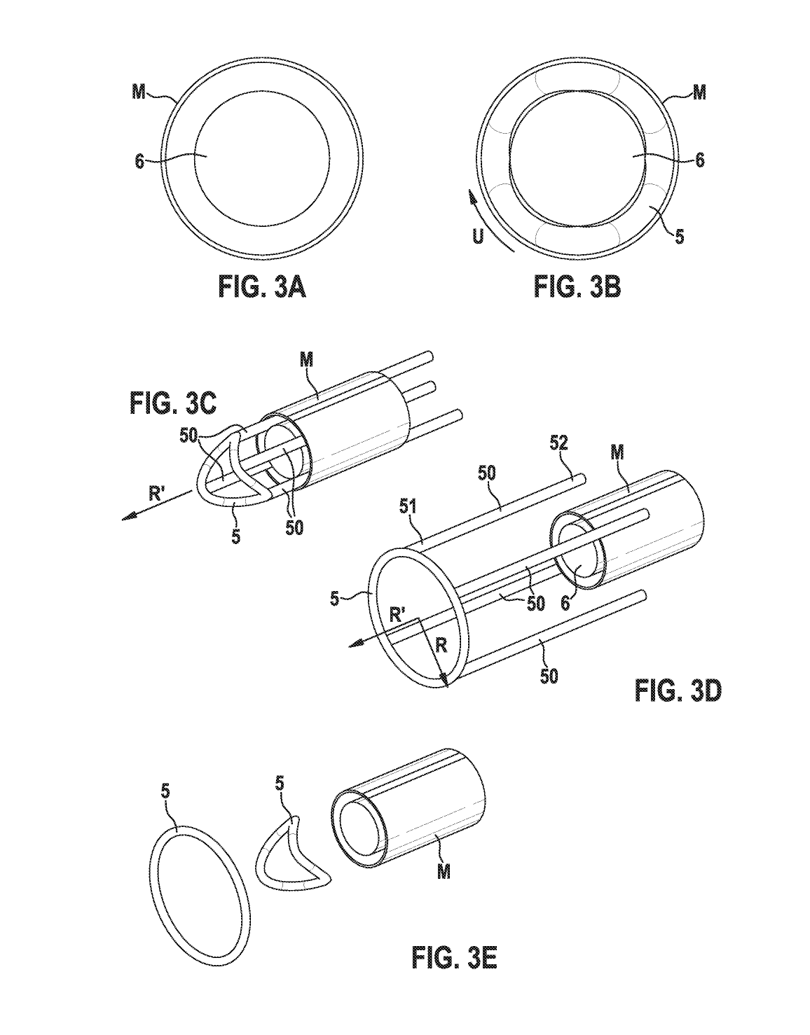

In accordance with FIGS. 3A-3E, the ring structure 5 can be formed as a closed, one-piece ring 5 formed from a self-expandable material. In FIGS. 3A-3E the area denoted by M shows the maximum diameter (here by way of example 18 F) which is not to be exceeded. The ring 5 in accordance with FIGS. 2C and 2E can be compressed to this diameter by arranging the four bars 50, which hold the ring 5, offset alternately axially to the front (in the distal direction R) and to the rear. Once the ring 5 has been released, it unfolds into its circle shape, as shown in accordance with FIGS. 2D and 2E. In accordance with FIG. 2D it can be seen that the bars 50 are fixed with their second end portions in the radial direction R close to the outer shaft 6, such that the bars 50 have a curved profile (in particular wound in an S shape).

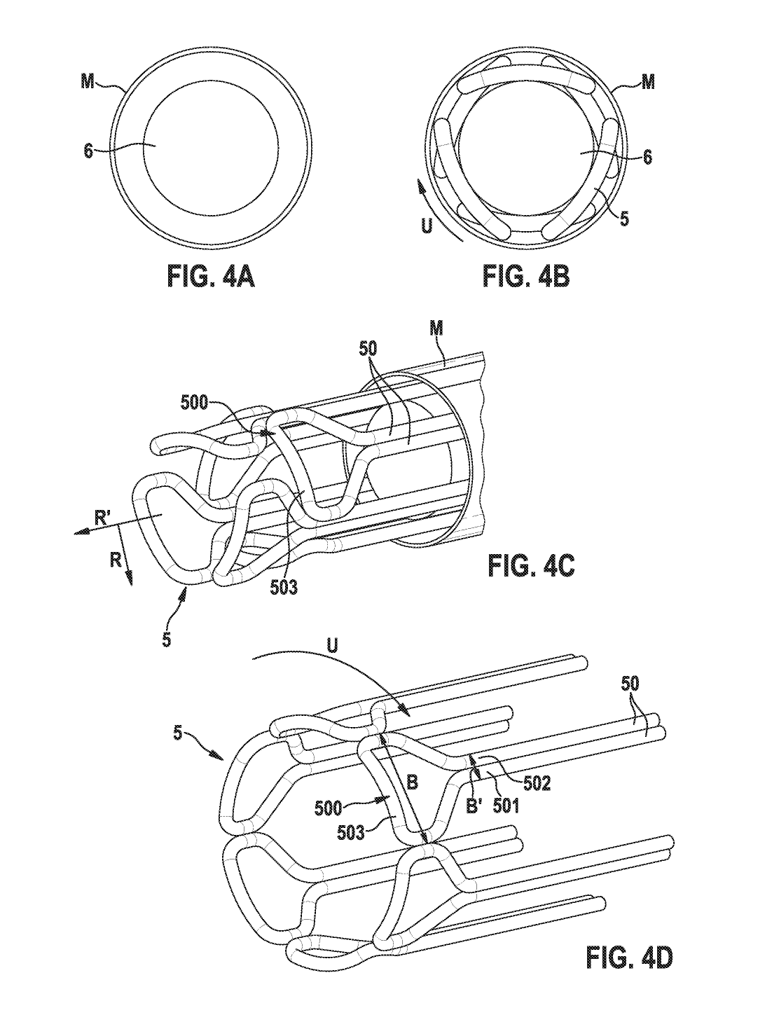

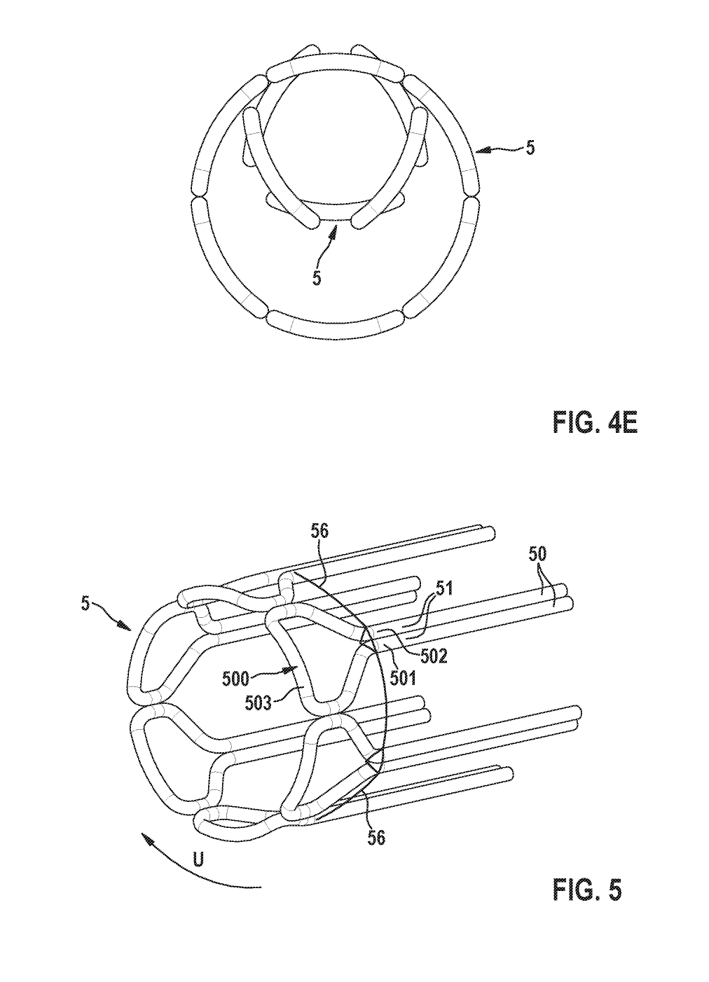

In accordance with an alternative embodiment the ring structure 5 according to FIGS. 4 and 5 can also be formed by a plurality of ring elements 500, wherein the ring elements 500 are movable relative to one another in such a way that the ring structure 5 is movable from a first configuration (see FIGS. 4C and 4E) into a second configuration (see FIGS. 4d and 4E), wherein the ring structure 5 in the second configuration (in relation to the radial direction R) has a greater diameter than in the first configuration.

The ring elements 500 are in particular formed in the manner of a closed bight in accordance with FIG. 4D. In other words, the ring elements 500 each have a curved portion 503, which extends from one end 501 of the ring element to another end 502 of the ring element 500, wherein the two ends 501, 502 in the circumferential direction U are opposite one another or are oriented in parallel and contact one another. The ring elements 500, away from the ends, hereby have a maximum width B in the circumferential direction U which is greater than the width B′ of the ring element 500 at the ends 501, 502. With a correspondingly offset arrangement of adjacent ring elements 500, the diameter of the ring structure 5 on the whole can hereby be reduced, as is shown in FIG. 4C, which shows the ring elements 500 or ring structure 5 in the first configuration. By contrast, FIG. 4D shows the ring structure 5 in the second configuration, in which the ring elements 500 are arranged in the same position and axially adjacently in the circumferential direction U, which results in the now larger diameter of the ring structure 5.

The bars 50 each adjoin the ends 501, 502 of the respective ring element 500 in one piece, such that two parallel bars 50 are connected by each ring element 500. Since the respective ring element 500 is connected integrally to the bars 50, the resultant structure 50, 500 does not have any sharp edges potentially endangering the heart valve prosthesis 2. In order to delimit the diameter of the ring structure 5, the individual ring elements 500 can each be fixed to the ring elements 500 adjacent in the circumferential direction U, for example by looping an elongate flexible element 56 through the ring elements 500, which flexible element in each case can be fixed in the region of the ends 501, 502, i.e. at the transition to the bars 50, as is shown in FIG. 5. The elongate element 56 can be a limp element, for example. The bars 50 can each extend along the outer shaft 6 to the stabilisation shaft 7 and can be fixed thereto, such that the ring structure 5 is movable by means of the stabilisation shaft 7, in particular is positionable in front of the capsule 4.

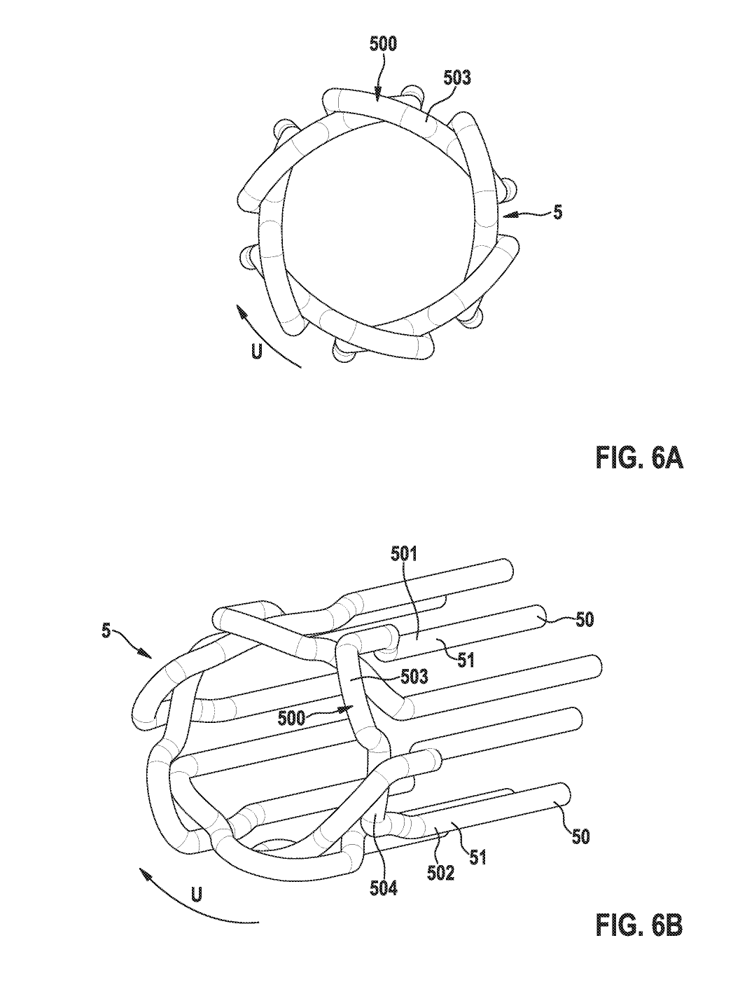

FIGS. 6A and 6B show an alternative embodiment of the ring elements 500, in which each two adjacent ring elements 500 engage in one another, such that the ring structure 5 is formed by a chain of the ring elements 500, wherein the ring elements 500 in the circumferential direction of the outer shaft 6 or the ring structure 5 are arranged such that they can be telescoped, such that the diameter of the ring structure is adjustable accordingly. The ring elements 500 are formed in the manner of an open bight. In other words, the respective ring element 500 has a portion 503 curved for example substantially in a U-shape, which extends from one end 501 of the ring element 500 to an opposite end 502 in the circumferential direction U, wherein here the respective two ends 501, 502 are arranged at a distance from one another. So that a substantially flush surface is present on the front side of the ring elements 500, the front sides of the portions 503 each have a step 504 (see FIG. 6B), over which an adjacent ring element 500 runs.

Also in the embodiment according to FIGS. 6A and 6B, the respective end 501, 502 is adjoined again in each case by a bar 50, which extends along the outer shaft 6, in particular to the stabilisation shaft 7 and is fixed thereto, such that the ring structure 5 is movable by means of the stabilisation shaft 7 or positionable in front of the capsule 4.

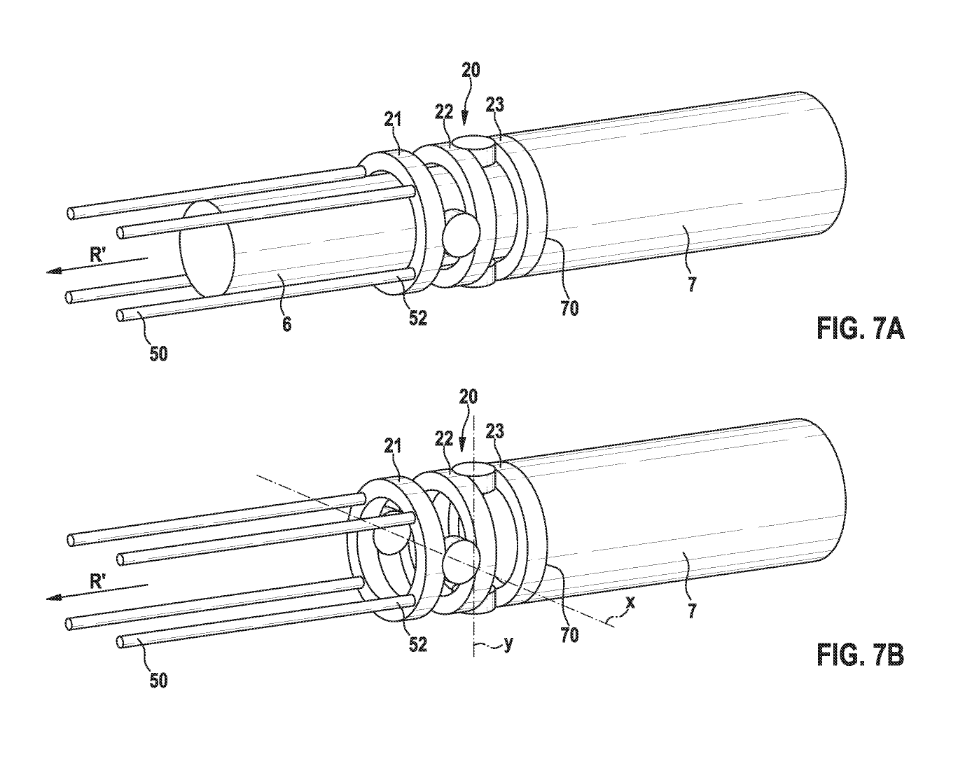

In accordance with the embodiment shown in FIGS. 7A and 7B of a catheter device 1 according to the invention, the bars can be connected by means of their second end portions 52, which are opposite the ring elements in the axial direction, via a joint 20 to the distal end 70 of the stabilisation shaft 7. The joint 20 is used to compensate deformations when the shafts of the catheter (for example in the aortic arch) are heavily curved. To this end, the joint 20 enables a connection of the bars 50 to the distal end of the stabilisation shaft 7 in a manner tiltable about two orthogonal axes x, y.

In accordance with the embodiment shown in FIGS. 7A and 7B, the joint 20 includes a first joint ring 21, a second joint ring 22, and a third joint ring 23, wherein the second joint ring 22 is arranged between the first and the third joint ring 21, 23. Here, the three joint rings 21, 22, 23 each surround the outer shaft 6. To produce the connection pivotable about the two orthogonal axes x, y, it is now provided that the first joint ring 21 is connected to the second joint ring 22 so as to be tiltable about a first axis x, the second joint ring in turn being connected to the third joint ring 23 so as to be tiltable about a second orthogonal axis y. Since the first joint ring 21 is rigidly connected to the second end portions 52 of the bars 50 and the third joint ring 23 is rigidly connected to the distal end 70 of the stabilisation shaft 7, a jointed fixing of the bars 50 to the distal end of the stabilisation shaft 7 is provided accordingly.

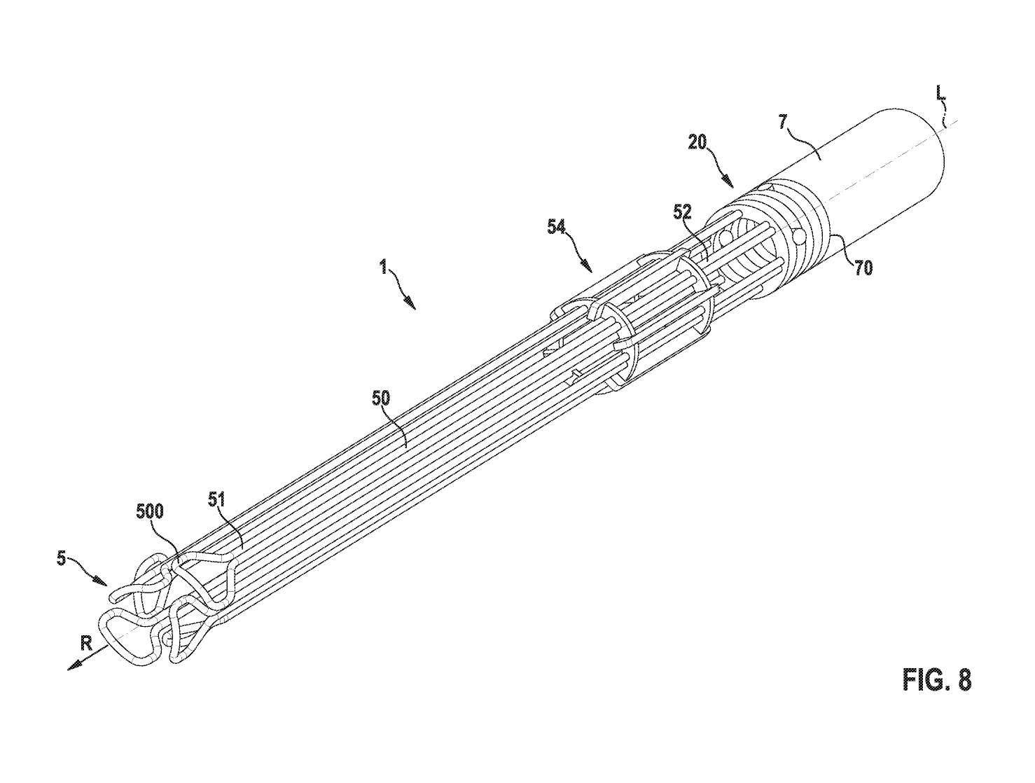

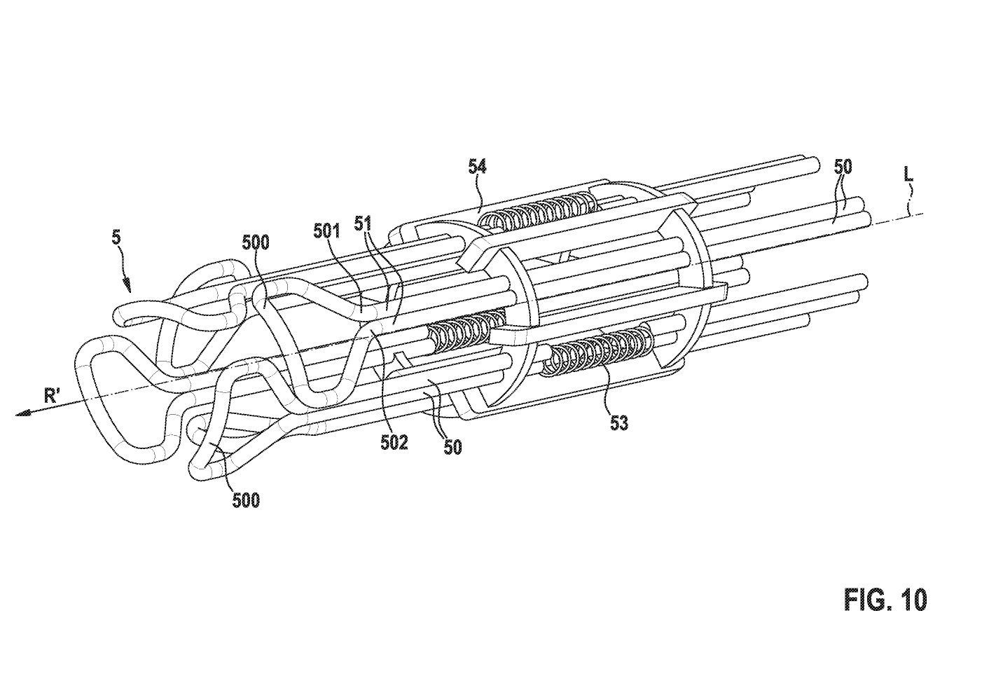

In the case of ring elements 500 that are arranged initially offset in alternation in the axial direction L or distal direction R′ (see above), it is also provided in accordance with FIGS. 8 and 10 that those ring elements 500 that are arranged further to the front in the distal direction R′ (see also FIG. 4C) are connected to bars 50 which have a portion 53 that is resiliently compressible in the axial direction L and that is formed in the present case in particular by a spring 53 in each case. In order to guide these bars 50, a guide 54 is furthermore provided, which surrounds the outer shaft 7, wherein in each case the portion of such a bar 50 extended between the spring 53 and the corresponding ring element 500 is guided through the guide 54, such that it can slide in the axial direction L in relation to the guide. The ring elements 500 or bars 50 not spring-loaded are by contrast coupled in particular rigidly to the guide 54.

If the ring structure 5 is now displaced in the distal direction R′ beyond the capsule 4, the ring elements 500 arranged further to the front, upon contact with the heart valve prosthesis 2 released in sections, are pushed back against a restoring force of the respective resiliently compressible portion (for example spring) 53 until all ring elements 500 contact the heart valve prosthesis 2. Here, there is automatically also a widening of the ring structure 5 to the larger diameter (second configuration, see also above).

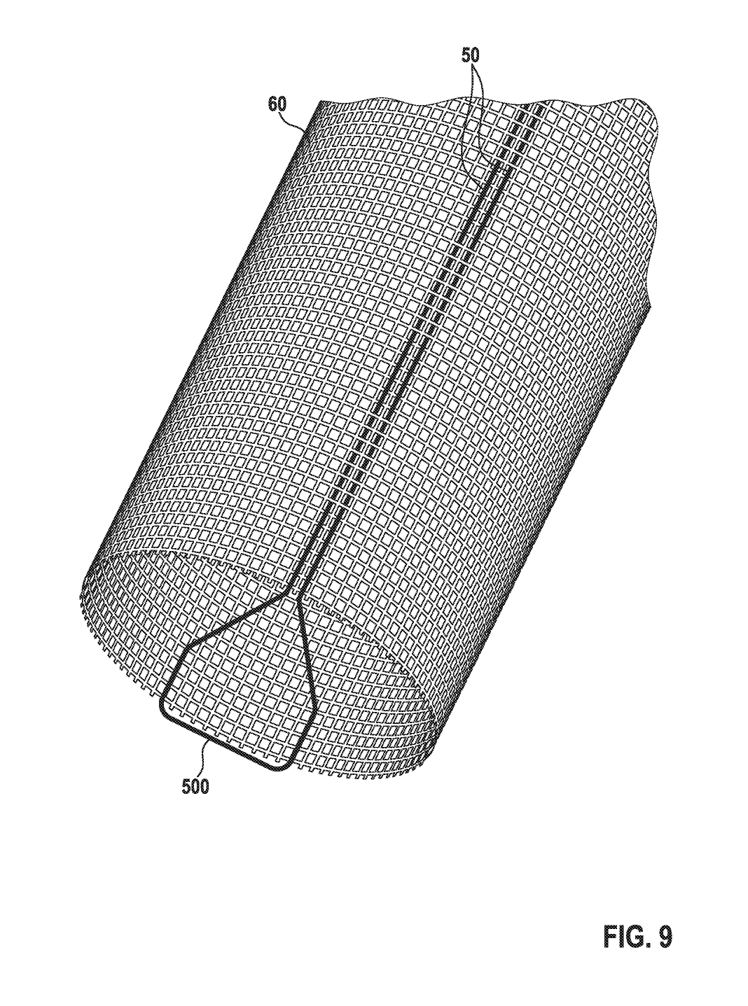

Lastly, the bars 50 can be surrounded by an outer jacket 60 surrounding the outer shaft 6, wherein the bars 50 can also be threaded into a jacket 60 of this kind in accordance with FIG. 9. The bars 50 here penetrate the jacket 60 at a number of points, such that they run alternately beneath and above the jacket 60. By means of the jacket 60, the diameter of the ring structure can thus be limited to a maximum diameter. A jacket of this kind can be made for example of a Dacron® fabric or another suitable material.

FIGS. 11A to 16D also show embodiments of the present invention which allow an active compression of the already partially expanded or released heart valve prosthesis 2 or of the stent framework 3.

The resheathing or re-insertion of a partially expanded portion T of a heart valve prosthesis 2 as shown in FIG. 13A-D in particular requires the diameter of the stent framework 3 to be significantly reducible (usually by a factor of 3 to 4), such that the heart valve prosthesis 2 can be received again in the capsule 4. In the prior art this is usually achieved by introducing an axial force (pulling), which loads the heart valve prosthesis 2 and the capsule 4 heavily and generates high frictional forces.

According to FIGS. 11A-C it is provided that the catheter device 1 has a ring structure 5 which is displaceable in the distal direction R′ or in the axial direction L, more specifically in particular over an already released, expanded portion of the heart valve prosthesis 2, such that this is compressible by means of the ring structure 5.

The ring structure 5 to this end, in accordance with FIGS. 11B and C. has a reducible diameter D′, such that the ring structure 5 is contractible about the partially unfolded heart valve prosthesis 2, and in so doing can exert a force in the radial direction R onto the heart valve prosthesis 2, which compresses the heart valve prosthesis 2, in particular the stent framework 3, in the radial direction R, such that the heart valve prosthesis 2 is retractable again into the capsule 4 (resheathing).

In particular, it is provided in accordance with FIGS. 11B and C that the ring structure 5 includes a plurality of bars 50 which extend in the distal direction R′ or axial direction L, and an elongate flexible element 560. The bars 50 each have a first end 50a, wherein the bars 50 at the first end 50a each have a through-opening 50b, and wherein the elongate flexible element 560 is guided through the through-openings 50b.

The element 560 can be a limp element. The element 560 can also be a wire, a thread, or a cord. The element 560 in particular forms a closed ring, such that a (synchronous and identically directed) rotation of the bars 50 about the longitudinal axis L′ of the bars 50 causes the flexible element 560 to be wound around the respective bar 50, such that the ring structure 5 is reduced in diameter D′, contracts around the partially expanded heart valve prosthesis 2, and compresses this in the radial direction R for re-insertion into the capsule 4.

The reduction of the diameter D′ with the rotation of the bars 50 is in particular generated in that the distance between adjacent bars 50 in the circumferential direction U of the ring structure 5 is reduced. The mechanism according to the invention here converts a torque/torsion into a movement of the ring structure 5 compressing the heart valve prosthesis 2 radially. In this way, high radial forces can be attained with comparatively low torques, since the lever arms are relatively short:

F=M/r

F: force in the circumferential direction between adjacent bars 50, which is converted into radial force.

M: torque acting on the respective bar 50.

r: radius of the respective bar 50 (lever arm).

Should the element 560 be loose, the bars 50, which in particular are flexible, can deform/bend in the radial direction (in relation to the middle line of the catheter or the longitudinal axis L), whereby the bars 50 are easily movable in the distal direction R′ over the capsule 4 and the partially expanded heart valve prosthesis 2 (see also FIG. 13C).

The bars 50 can be displaceable in particular by means of the stabilisation shaft 7 in the distal direction R′. The bars 50 can also be coupled via their second ends, which are opposite the first ends 50a, to a mechanism by means of which the bars 50 are rotatable about the respective longitudinal axis L′ (synchronously and each in the same direction).

As is shown in FIGS. 11A-11C, the first ends 50a are preferably spherical, whereby potential damage to the heart valve prosthesis 2 is prevented by the first ends 50a.

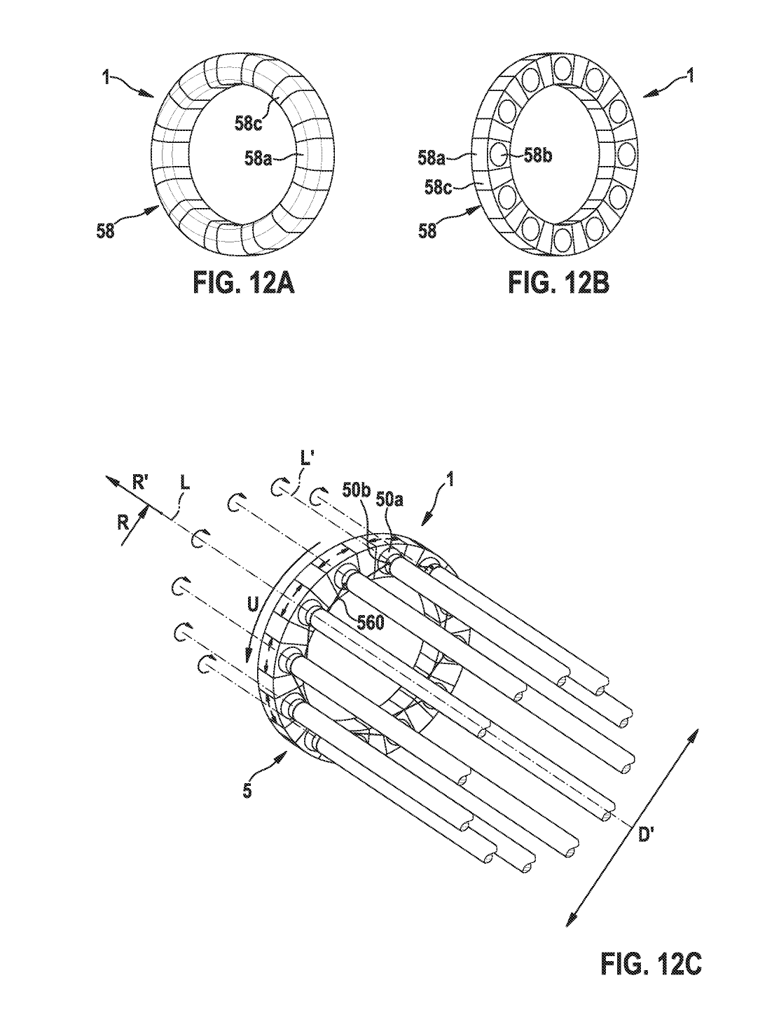

In accordance with FIGS. 12A-12C the first ends 50a can be mounted in an annular element 58 of the ring structure 5, wherein the first ends 50a of the bars 50 in each case engage in an associated recess 58b of the annular element 58 and are mounted there rotatably about the longitudinal axis L′ of the respective bar 50. The respective recess 58b here forms a rotary bearing or plain bearing for the first end 50a of the respective bar 50.

The recesses or plain bearings 58b can be arranged according to FIGS. 12B and 12C in each case in a rigid portion 58a of the annular element 58, wherein each two rigid portions 58a adjacent in the circumferential direction U can be connected to one another by an intermediate portion 58c, which for example is resiliently deformable. By rotating the bars 50, the resiliently deformable intermediate portions 58c can be pressed together in the circumferential direction U of the annular element 58, wherein the diameter D′ of the annular element 58 is reduced. Here as well, specifically, on account of the flexible element 560 guided annularly through the through-openings 50b the bars 50 are moved closer to one another in the circumferential direction U when the bars 50 are rotated synchronously about their respective longitudinal axis L′ (in the same direction).

The intermediate portions 58c furthermore allow an increase in the diameter D′ of the ring structure 5 or of the annular element 58, specifically if the ring structure 5, when the element 560 is loose, is slid over the capsule 4 in the distal direction R′ so as to surround the partially expanded heart valve 2 in order to compress the heart valve 2. A subsequent rotation of the bars 50 (see above) then causes the ring structure 5 or the annular element 58 to contract around the partially expanded heart valve prosthesis 2 (or the stent framework 3) and to compress this in the radial direction for re-insertion into the capsule 4.

The intermediate portions 58c in particular constitute a mechanical coupling/connection of the rigid portions 58a or bearings 58b, such that the rigid portions 58a/bearings 58b are movable relative to one another in the circumferential direction U of the ring structure 5. Regardless of the above-described resiliently deformable intermediate elements 58a, the intermediate elements 58c can be formed in particular as spring elements or as elements that allow at least or only a sliding movement of the rigid portions 58a/bearings 58b relative to one another in the circumferential direction U. The intermediate portions 58c can be made in particular from an anisotropic material, which in particular is flexible only in the circumferential direction U, but not in the other directions (for example transversely to the circumferential direction U).

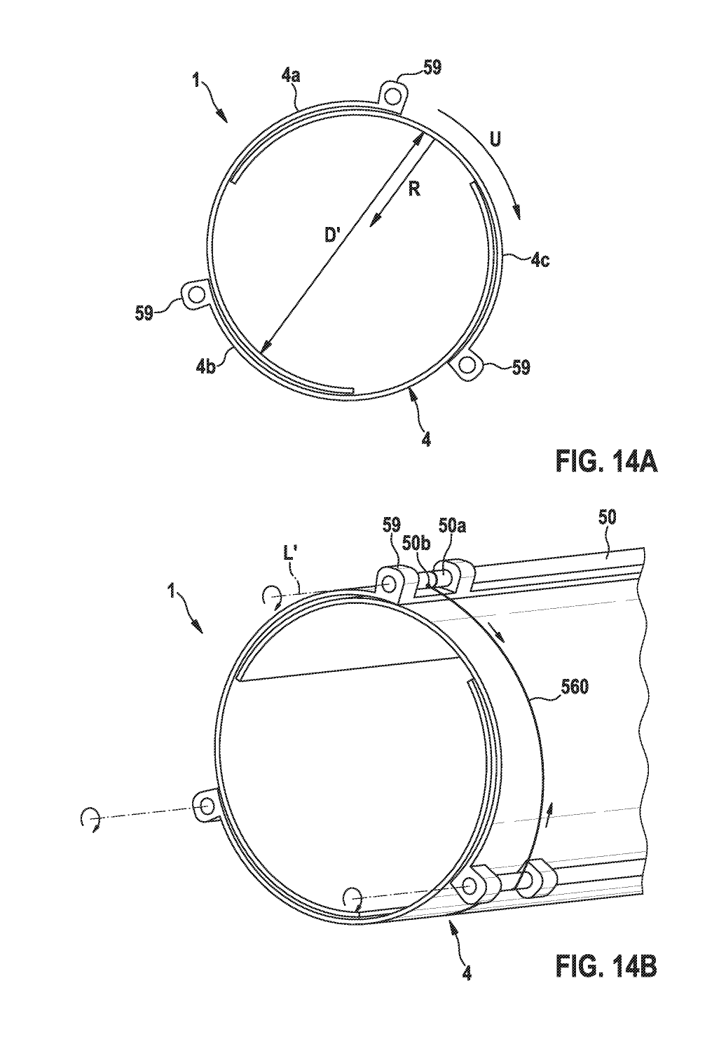

FIGS. 14A-14B also shows an embodiment in which the capsule 4 itself forms a ring structure of actively variable diameter D′. Here, in contrast to the other embodiments, there is thus no additional ring structure 5 slid over the capsule 4 and over the partially expanded heart valve prosthesis 2, but instead a corresponding mechanism is integrated in the capsule 4.

The capsule 4 in this example includes three (for example convexly curved or shell-shaped) wall elements 4a, 4b, 4c, wherein wall elements 4a, 4b, 4c that are adjacent in the circumferential direction U of the capsule 4 overlap one another, such that a circumferential wall structure is provided. Furthermore, each wall element 4a, 4b, 4c includes a bearing 59 for a first end portion 50a of a bar 50, which extends in the distal direction R′ or axial direction L, wherein each bar 50 is rotatable in its bearing 59 about its longitudinal axis L′. The bars 50 again each include a through-opening 50b, through which an elongate flexible element 560 is guided.

The element 560 can again be a limp element. The element 560 can also be a wire, a thread, or a cord. The element 560 in particular forms a closed ring, such that a (synchronous and identically directed) rotation of the bars 50 about the longitudinal axis L′ of the bars 50 causes the flexible element 560 to be wound around the respective bar 50, such that each two wall elements 4a, 4b, 4c overlapping one another are slid one over the other in the circumferential direction U, wherein the capsule or ring structure 4 comprising the wall elements 4a, 4b, 4c is reduced in diameter and in so doing contracts around the partially expanded heart valve prosthesis and compresses this in the radial direction R for reinsertion into the capsule 4 (see FIG. 14A). The bars 50 can also be coupled via their second end portions (not shown), which are opposite the first end portions 50a, to a mechanism by means of which the bars 50 are rotatable about the respective longitudinal axis L′.

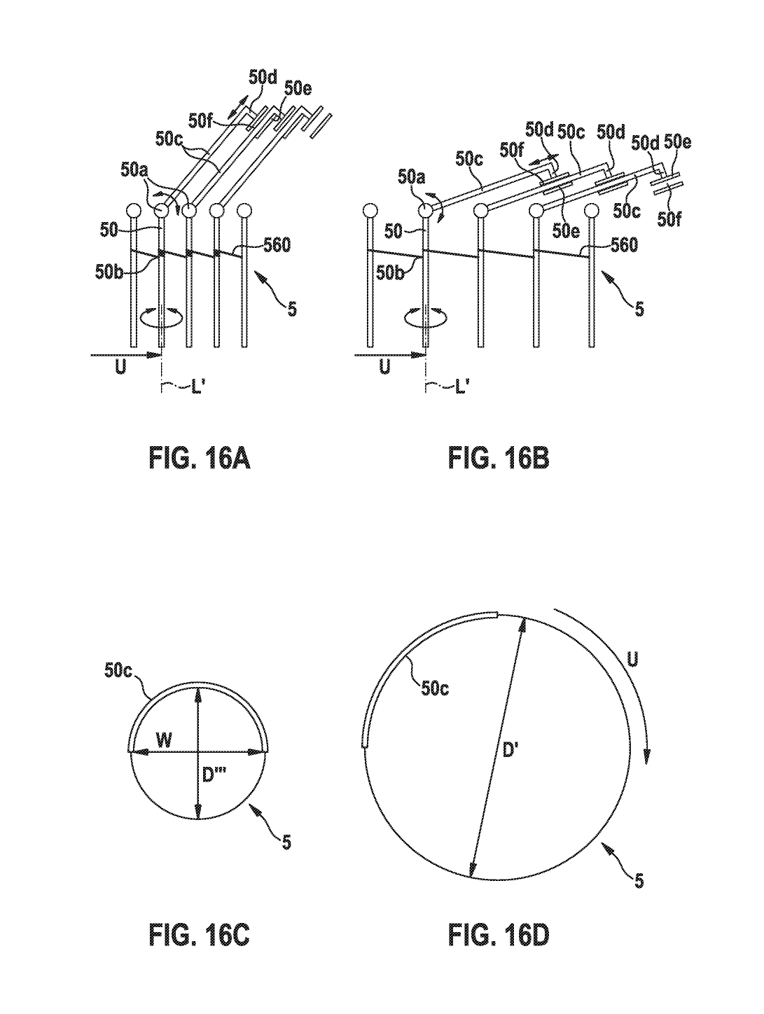

FIGS. 1A-15D5, in conjunction with FIGS. 16A-16D, shows a further embodiment of a ring structure 5 of reducible diameter D′ for radial compression of a partially expanded heart valve prosthesis 2.

Here it is provided that the ring structure 5 includes a plurality of bars 50 which extend in the distal direction R′ or axial direction L, and an elongate flexible element 560. The bars 50 each have a first end 50a, at which the respective bar 50 has a through-opening 50b, wherein the elongate flexible element 560 is guided through the through-openings 50b. The first ends 50a can each be spherical.

Furthermore, each first end 50a is connected to a helically curved connection bar 50c (in particular in one piece), wherein the respective connection bar 50c has an angled end portion 50d, which ends in a spherical end 50e, which has a through-opening 50f formed as a plain bearing, wherein an adjacent connection bar 50c is mounted slidingly in the respective through-opening 50f.

The elongate, flexible element 560 can again be a limp element 560. The element 560 can furthermore be a wire, a thread, or a cord. The element 560 in particular forms a closed ring, such that a rotation of the bars 50 about the longitudinal axis L′ of the bars 50 causes the flexible element 560 to wind around the respective bar 50.

The bars 50 here move closer together in accordance with the two-dimensional projection shown in FIG. 16 (A and B) (change from FIG. 14B to FIG. 14C), wherein the plain bearings 50f of the connection bars 50c are displaced towards the angled end portion of the guided connection bar 50c. Accordingly, the diameter D′ of the ring structure reduces to the smaller diameter D″, as is shown in FIG. 16C.

The bars 50 or connection bars 50c can thus contract around the partially expanded heart valve prosthesis 2 by (synchronous and identically directed) rotation of the bars 50 about their longitudinal axes L′, wherein the heart valve prosthesis 2 is compressed in the radial direction R for re-insertion into the capsule 4. An opposite rotation accordingly allows an enlargement of the diameter of the ring structure 5 (for example in order to slide this in the distal direction R′ beyond the capsule 4 onto the partially expanded heart valve prosthesis 2).

The bars 50 can also be coupled via their second end portions (not shown), which are opposite the first end portions 50a, to a mechanism by means of which the bars 50 are rotatable about the respective longitudinal axis L′.

The spherical ends 50a and 50e can be formed by spheres which for example are connected by a press connection or an adhesive connection to the respective bar 50 or the respective connection bar 50c.

The spheres can be formed for example from a sapphire, a ruby or a ceramic. The bars and connection bars preferably consist of a metal, such as stainless steel. The spheres can have a diameter of 0.9 mm, for example. The diameter of the connection bars can be 0.5 mm, for example. The helical connection bars for example can have a length that is equal to a corresponding circle sector angle, such as W=180°, when the ring structure 5 has a minimal diameter D″ (for example in accordance with the example in FIG. 16C). The length of the connection bars 50c can vary and is selected such that a desired diameter reduction of the ring structure 5 can be provided.

It will be apparent to those skilled in the art that numerous modifications and variations of the described examples and embodiments are possible in light of the above teaching. The disclosed examples and embodiments are presented for purposes of illustration only. Therefore, it is the intent to cover all such modifications and alternate embodiments as may come within the true scope of this invention.

1. A catheter device for implanting a self-expanding implant, said catheter device comprising:
a self-expandable framework,
an outer shaft, which has a capsule at a distal end, which capsule surrounds the framework, wherein the capsule and framework are displaceable relative to one another, such that the framework is releasable in sections, wherein a released portion of the framework expands automatically and can be re-introduced into the capsule, and
a ring structure positionable over an already released, expanded portion of the framework such that the already released, expanded portion of the framework is contactable and/or compressible by the ring structure.

2. The catheter device according to claim 1, wherein the ring structure is configured to limit a diameter of the framework at the location of the ring structure, such that an axial force which is exerted onto the framework by the capsule as the framework is resheathed into the capsule is limited.

3. The catheter device according to claim 1, comprising a plurality of bars for supporting the ring structure, which bars are each connected to the ring structure via a first end portion.

4. The catheter device according to claim 1, comprising an outer stabilisation shaft for stabilising the outer shaft, wherein the outer shaft is arranged in a lumen of the stabilisation shaft surrounded by the stabilisation shaft, and wherein the outer shaft and the stabilisation shaft are displaceable relative to one another.

5. The catheter device according to claim 4, wherein the bars are each fixed via a second end portion to a distal end of the stabilisation shaft.

6. The catheter device according to claim 5, wherein the second end portions of the bars are connected via a joint to the distal end of the stabilisation shaft.

7. The catheter device according to claim 6, wherein the joint has a first joint ring, a second joint ring, and a third joint ring, wherein the second joint ring is arranged between the first and the third joint ring, and wherein the three joint rings surround the outer shaft, wherein the first joint ring is connected to the second joint ring so as to be tiltable about a first axis (x), and wherein the second joint ring is connected to the third joint ring so as to be tiltable about a second axis (y), wherein the two axes (x, y) run orthogonally to one another, and wherein the first joint ring is rigidly connected to the second end portions of the bars, and wherein the third joint ring is connected to the distal end of the stabilisation shaft.

8. The catheter device according to claim 1, wherein the ring structure is a continuous, closed, self-expanding ring.

9. The catheter device according to claim 1, wherein the ring structure comprises a plurality of ring elements that are movable relative to one another in such a way that the ring structure is movable from a first configuration into a second configuration, wherein the ring structure in the second configuration has a larger diameter than in the first configuration.

10. The catheter device according to claim 9, comprising a plurality of bars for supporting the ring structure, which bars are each connected to the ring structure via a first end portion, wherein each bar is connected via its first end portion to a ring element (500).

11. The catheter device according to claim 9, wherein each of two adjacent ring elements engage in one another.

12. The catheter device according to claim 9, wherein each of two adjacent ring elements in the first configuration are arranged axially offset in relation to one another, such that the ring structure has first ring elements which in the first configuration of the ring structure are arranged in the distal direction (R′) in front of second ring elements of the ring structure, and in that in the second configuration the ring elements are arranged adjacently in the circumferential direction (U).

13. The catheter device according to claim 12, comprising bars for supporting the ring structure and connected to the first ring elements that have a portion that is resiliently compressible in the axial direction, such that, when the ring structure is displaced in the distal direction (R′), the first ring elements, when they contact the partially released framework, are slid back against a restoring force of the particular resiliently compressible portion until all ring elements contact the framework.

14. The catheter device according to claim 13, wherein the bars are surrounded by an outer jacket, wire or tape material surrounding the outer shaft or are fixed to an outer jacket, wire or tape material surrounding the outer shaft.

15. The catheter device according to claim 1, wherein the framework comprises a stent and the stent supports a heart valve prosthesis.
